(19) United States
(12) Patent Application Publication
(10) Pub. No.: US 2017/0368024 A1
(43) Pub. Date: Dec. 28, 2017

(54) Compositions and Methods for Treating Glaucoma

(71) Applicant: ORANGE COUNTY GLAUCOMA, Santa Ana, CA (US)

(72) Inventor: Richard A. Hill, Santa Ana, CA (US)

(21) Appl. No.: 15/540,299

(22) PCT Filed: Dec. 28, 2015
(86) PCT No.: PCT/US15/67731

§ 371 Date: Jun. 28, 2017

Related U.S. Application Data
(60) Provisional application No. 62/097,452, filed on Dec. 29, 2014.

(51) Int. Cl. A61K 31/375; A61K 9/00; A61K 31/7088; A61K 38/43; A61K 35/30; A61K 9/08

(52) U.S. Cl. CPC A61K 31/375; A61K 38/43; A61K 35/30; A61K 9/0048; A61K 31/7088; A61K 9/0051; A61K 9/08

(57) ABSTRACT

Compositions and methods for treating glaucoma by restoring the filtration capabilities of the endothelial lining of Schlemm's canal are provided. More particularly, methods of lowering intraocular pressure are disclosed, where a subject in need of such treatment is administered a therapeutically effective amount of a composition. In various embodiments, the composition may include tyrosine or L-DOPA conjugated to an ascorbic acid, an enzyme, a nucleic acid encoding an enzyme, or trabecular meshwork cells.

### BACKGROUND

### Field

Aspects of the disclosure relate generally to methods and compositions for treating glaucoma. More particularly, the treatment of glaucoma may involve restoring the filtration capability of the trabecular meshwork of the eye.

### Description of the Related Art

Glaucoma is a leading cause of blindness characterized by increased pressure within the eye, which, if untreated, can lead to destruction of the optic nerve. A clear fluid called aqueous humor is formed constantly by the ciliary bodies and secreted into the posterior chamber. This fluid passes over the lens and enters the anterior chamber. Aqueous humor passes out the anterior chamber of the eye at approximately the same rate at which it is produced through one of two routes. Approximately 10% of the fluid percolates between muscle fibers of the ciliary body, and approximately 90% of the fluid is removed via the “canalicular route,” through a filter-like mass of tissue called the trabecular meshwork and Schlemm's canal, and then enters the scleral venous network.

There are a number of different forms of glaucoma, including open-angle and closed-angle glaucoma, as well as steroid induced glaucoma. The most common form of glaucoma is open-angle, which results from increased resistance in the outflow pathway through the trabecular meshwork. The mechanism by which the outflow pathway becomes blocked or inadequate is poorly understood, but the result is an increase in pressure within the eye, which compresses the axons in the optic nerve and can compromise vascular supply to the nerve. Over time, this can result in partial or total blindness. The trabecular meshwork is not physically obstructed, but no longer efficiently transports fluid between the anterior chamber and the scleral drainage veins.

Current treatment of glaucoma is either medical, surgical, or both. Medications for the treatment of glaucoma include prostaglandin analogs, which increase fluid percolation between muscle fibers of the ciliary body, and miotics, which are administered as drops and cause contraction of the pupil of the eye by tightening the muscle fibers of the iris to increase the rate at which the aqueous humor leaves the eye. Epinephrine drops have also been successful in reducing intraocular pressure, but have significant side effects. Other medications are employed, such as β-adrenergic blocking agents, as drops, or carbonic anhydrase inhibitors as pills, which reduce the production of fluid.

Surgical solutions include applying a laser to multiple spots along the trabecular meshwork, which is thought to change the extracellular material and enhance outflow. Approximately 80% respond initially to this treatment, but, unfortunately, 50% have increased pressure within five years. Other solutions attempt to increase the permeability of the trabecular meshwork or widen Schlemm's canal. Another surgical procedure is a trabeculectomy, wherein an incision is made in the conjunctiva to form a hole in the sclera for aqueous fluid to flow through. This can be performed either with a laser or through an open procedure. Both routes have risks, including infection or injury to the eye. With either route, frequently the hole closes up over time with consequent increase in pressure. A variety of apparatuses have been suggested, such as implantation as a shunt or drain across the trabecular network, draining either to the sclera or to Schlemm's canal. Alternatively, some treatments have targeted the pores between endothelial cells lining Schlemm's canal.

However, a need remains for a way of safely, lastingly, and effectively treating open-angle glaucoma. Current medical and surgical treatment options often lose their efficacy with time. Furthermore, surgical treatments have associated risks of infection or injury to the eye, and current medical solutions often come with significant side effects either affecting vision, the structures of the eye, or with systemic side effects. A need also exists for a treatment of glaucoma which addresses the underlying pathology in the aqueous humor outflow system and leads to a return of drainage as seen in non-glaucomatous eyes. There exists a need as well for improved models of testing drugs ex vivo for use in this ophthalmic application.

### SUMMARY

Methods and compositions for the treatment of glaucoma are provided. The disclosure is based on the discovery of ascorbic acid conjugates in ocular tissue, which provides a potential mechanism for regulation of intraocular pressure.

A method of lowering intraocular pressure is disclosed. The method includes administering to a subject in need of such treatment, a therapeutically effective amount of an ascorbic acid conjugate.

In one embodiment, the ascorbic acid conjugate comprises ascorbic acid or a derivative thereof coupled to tyrosine or a derivative thereof. In other embodiments, the ascorbic acid conjugate comprises ascorbic acid or a derivative thereof coupled to L-DOPA or a derivative thereof.

In one embodiment, a method of lowering intraocular pressure includes administering to a subject in need of such treatment, a therapeutically effective amount of a composition that increases an enzymatic activity of conjugating ascorbic acid to tyrosine or a derivative thereof, or to L-DOPA or a derivative thereof. The composition may be an enzyme or enzymatically active fragment thereof comprising kinase activity. In other embodiments, the composition may be an enzyme or enzymatically active fragment thereof comprising esterase activity. In some embodiments, the composition is a nucleic acid that encodes for an enzyme or enzymatically active fragment thereof.

A pharmaceutical composition for use in lowering intraocular pressure is also disclosed. The composition comprises ascorbic acid conjugated to tyrosine or L-DOPA or derivatives thereof, and a pharmaceutically acceptable carrier, wherein the pharmaceutical composition is configured for intraocular delivery.

In a variation, the pharmaceutical composition for use in lowering intraocular pressure comprises an enzyme or enzymatically active fragment capable of catalyzing the conjugation of ascorbic acid to tyrosine or L-DOPA or derivatives thereof, and a pharmaceutically acceptable carrier, wherein the pharmaceutical composition is configured for intraocular delivery.

In another variation, the pharmaceutical composition for use in lowering intraocular pressure comprises an enzyme or enzymatically active fragment capable of catalyzing the linking of an ascorbic acid conjugate to a transmembrane protein, and a pharmaceutically acceptable carrier, wherein the pharmaceutical composition is configured for intraocular delivery, and wherein the conjugate comprises ascorbic acid or a derivative thereof coupled to tyrosine or L-DOPA or derivatives thereof.

In another variation, the pharmaceutical composition for use in lowering intraocular pressure comprises a nucleic acid which encodes an enzyme or enzymatically active fragment capable of catalyzing the conjugation of ascorbic acid to tyrosine or L-DOPA or derivatives thereof, and a pharmaceutically acceptable carrier, wherein the pharmaceutical composition is configured for gene therapy.

In another variation, the pharmaceutical composition for use in lowering intraocular pressure comprises a nucleic acid which encodes an enzyme or enzymatically active fragment capable of catalyzing the linking of an ascorbic acid conjugate to a transmembrane protein, and a pharmaceutically acceptable carrier, wherein the pharmaceutical composition is configured for intraocular delivery, and wherein the conjugate comprises ascorbic acid or a derivative thereof coupled to tyrosine or L-DOPA or derivatives thereof.

Another method of lowering intraocular pressure is also disclosed. The method includes administering to a subject in need of such treatment, trabecular meshwork cells to an anterior chamber of an eye.

In one embodiment, the trabecular meshwork cells may be administered in free solution.

Another method of lowering intraocular pressure is also disclosed. The method includes administering to a subject in need of such treatment, trabecular meshwork cells onto a trabecular meshwork of an eye.

In one embodiment, the trabecular meshwork cells may be administered under direct visualization, in another embodiment, the trabecular meshwork cells may be administered under indirect visualization. In some embodiments, the trabecular meshwork cells may be cultured cells from suspension, cell or organ cultures. In some embodiments, prior to administration the trabecular meshwork cells may be genetically modified in vitro to correct a hereditary or acquired defect.

A composition for use in lowering intraocular pressure is also disclosed. The composition comprises trabecular meshwork cells in a pharmaceutically acceptable carrier, wherein the composition is configured for intraocular delivery or delivery to a region of the trabecular meshwork.

### BRIEF DESCRIPTION OF THE DRAWINGS

FIG. 1A is a cross-sectional illustration of the anterior portion of the eye.

FIG. 1B is a cross-sectional illustration of the irido-corneal angle of the eye.

FIG. 2A is a schematic of the trabecular meshwork and Schlemm's canal; FIG. 2B-C are schematics of the membrane of an endothelial cell in the juxtacanalicular lining of Schlemm's canal.

FIG. 3 is an illustration of proposed structures of ascorbate conjugates for tyrosine and L-DOPA.

FIG. 4 is a graph showing a trabecular meshwork sample extract L/MS/MS of 339.10/176.1 u.

FIG. 5 is a graph showing a trabecular meshwork sample extract L/MS/MS of 355.09/176.1 u.

FIG. 6 is a graph showing a trabecular meshwork sample extract LC/MS/MS.

### DETAILED DESCRIPTION

### Anatomy

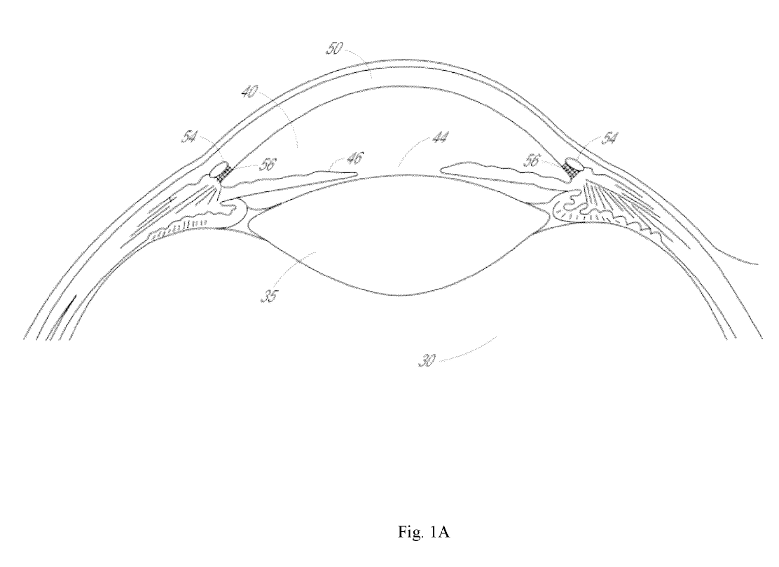

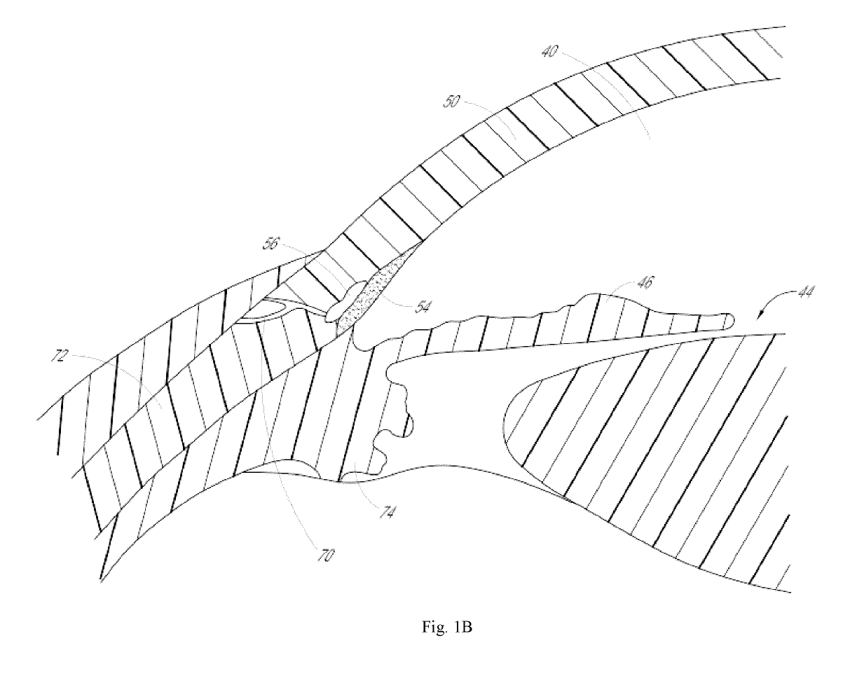

Glaucoma is defined by increased pressure in the chambers of the eye resulting from disordered drainage of the aqueous humor from the anterior chamber 40 (FIG. 1A) of the eye into the aqueous veins 70 (FIG. 1A) and thence to the sacral venous drainage system. The precise mechanism of drainage is poorly understood. However, it is known that the process, in a normal eye, is energy independent and self-regulating, such that the pressure of the eye remains relatively constant. The outflow rate from the anterior chamber of the eye generally matches the production rate of aqueous humor in the posterior chamber of the eye 30.

In the normal eye (FIGS. 1A and 1B), aqueous humor flows through the trabecular meshwork 54 into Schlemm's canal 56, and thereby into the venous system 60 of the sclera 72. The trabecular meshwork 54 and Schlemm's canal 56 are located at the junction between the iris 46 and the sclera 72. The cornea 50, lens 35, and pupil 44 are also visualized. The trabecular meshwork is wedge shaped in structure and runs around the entire circumference of the eye, forming a three dimensional sieve structure. The trabecular meshwork is formed of collagen beams aligned with a monolayer of cells called the trabecular cells, which produce an extracellular substance which fills the spaces between collagen beams. After passing through the trabecular meshwork, aqueous matter crosses the endothelial cells of the Canal of Schlemm 56. In this manner, trabecular meshwork cells and Canal of Schlemm endothelial cells are thought to comprise the cells of the primary outflow pathway of the eye. The trabecular meshwork is suspended between the corneal endothelium and the ciliary body face and is comprised of a series of parallel layers of thin, flat, branching and interlocking bands termed trabeculae. The inner portion of the trabecular meshwork (closest to the iris root and ciliary body 74) is called the uveal meshwork, whereas the outer portion (closest to the Canal of Schlemm) is called the corneoscleral or juxtacanalicular meshwork. The uveal meshwork trabeculae measure approximately 4 μm in diameter, consist of a single layer of cells surrounding a collagen core, and are arranged in layers which are interconnected. The spaces between these trabeculae are irregular and range from about 25 μm to about 75 μm in size. The trabeculae of the corneoscleral meshwork resemble broad, flat endothelial sheets about 3 μm thick and up to about 20 μm long. The spaces between these trabeculae are smaller than in the uveal meshwork and more convoluted. As the lamellae approach the Canal of Schlemm, the spaces between the trabeculae decrease to about 2 μM. The resistance to aqueous humor outflow through the trabecular meshwork has been reported to reside primarily in the juxtacanalicular meshwork (JCM). At this site two cell types are found: trabecular meshwork cells and also endothelial cells of the inner wall of Schlemm's canal. Treatments, both medical and surgical, have attempted to reduce intraocular pressure by increasing the permeability of the trabecular meshwork, creating new outflow pathways, or widening Schlemm's canal. However, these do not adequately address the juxtacanalicular meshwork as the primary source of resistance to outflow.

In contrast to the current level of knowledge regarding cellular processes responsible for aqueous humor production by the ciliary body 74, relatively little is known about the cellular mechanisms in the trabecular meshwork 54 that determine the rate of aqueous outflow. Pinocytotic vesicles have been observed in the juxtacanalicular meshwork and the inner wall of Schlemm's Canal. The function of these vesicles remains unknown, but some investigators have suggested that the bulk flow of aqueous humor through the meshwork cannot be accounted for by flow through the intercellular spaces and that these vesicles play a central role in outflow regulation. Management of outflow by regulation of ion channels in the cell membranes of the juxtacanalicular meshwork and lining of Schlemm's Canal has been proposed. However, it is proposed that a different mechanism, an osmotic drive, is responsible for the regulation of outflow of aqueous humor through the JCM. This osmotic drive is self-regulating, such that changes in intraocular pressure lead to corresponding changes in the rate of outflow so that a relatively constant pressure is maintained.

### Experiment

It is known that the levels of L-ascorbic acid in the aqueous humor (1.06 mmol/l; Arshinoff S. A., et al. “Ophthalmology”, chapter 4.20.2, published by Mosby International Ltd., 1999, herein incorporated by reference in its entirety) are about 20 times higher (Brubaker R. F. et al. “Investigative Ophthalmology Visual Science”, June 2000, vol. 41, No. 7, pp. 1681, herein incorporated by reference in its entirety) than those present in the blood circulation (20-70 μmol/l, Geigy Scientific Tables, vol. 3, page 132, 8th edition 1985, published by Ciba Geigy, herein incorporated by reference in its entirety). In the case of the retina, the levels of L-ascorbic acid in the eye are actually 100 times higher than those present in the blood circulation.

Studies investigating the levels of ascorbic acid in the glaucomic eye (Pei-fei Lee, M D et al., “Aqueous Humor Ascorbate Concentration and Open-Angle Glaucoma,” Arch Ophthalmol. 1977; 95(2):308-310, herein incorporated by reference in its entirety) and assessing the use of dietary antioxidants in preventing glaucoma (Jae H. Kang et al., “Antioxidant Intake and Primary Open-Angle Glaucoma: A Prospective Study,” Am. J. Epidemiol. (2003) 158 (4): 337-346, herein incorporated by reference in its entirety) show that the level of ascorbic acid did not appear to be predictably reduced in the glaucomic eye, nor does antioxidant use prevent glaucoma. Treatments directed at use of ascorbic acid supplements have been proposed, theorizing that the antioxidant properties may play a role in maintaining reduced intraocular pressure (US2006/0004089, herein incorporated by reference in its entirety).

However, there has not been a satisfactory explanation for the increased levels of L-ascorbic acid in the eye nor an explanation of the role that it plays in maintaining normal function of the eye.

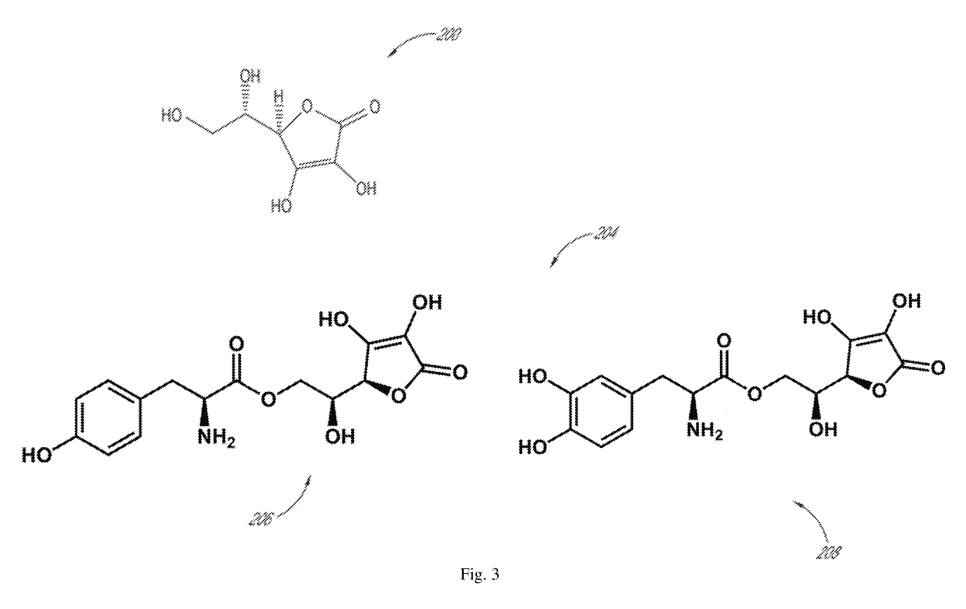

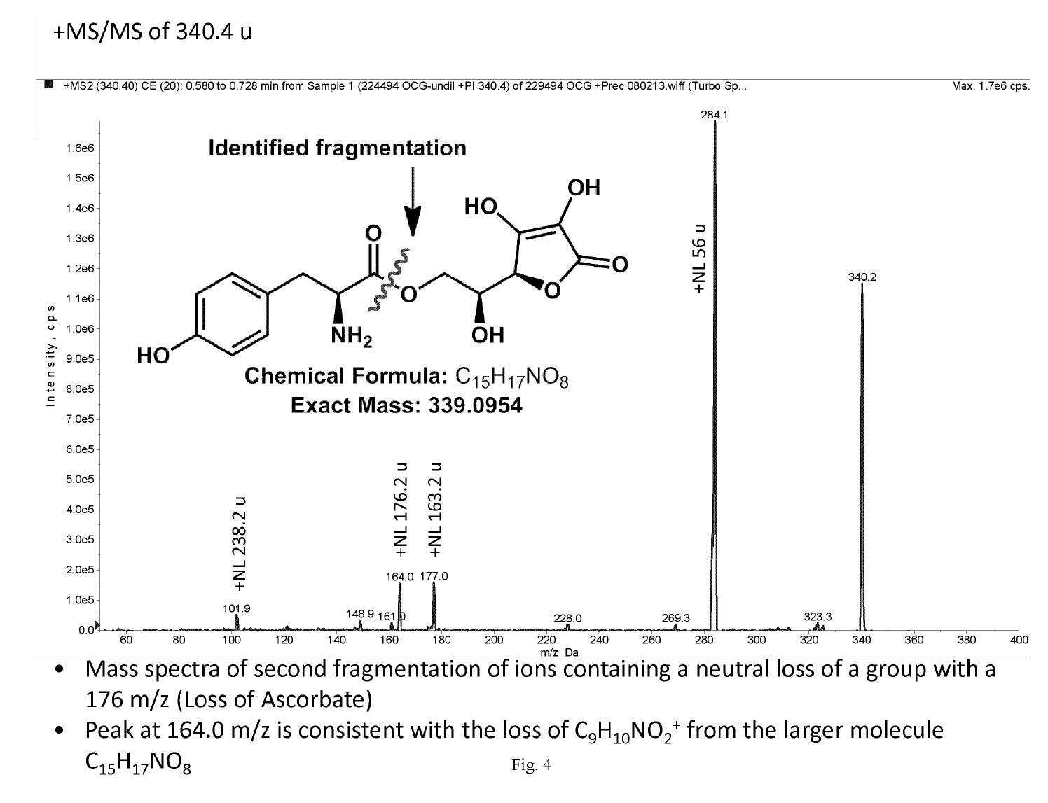

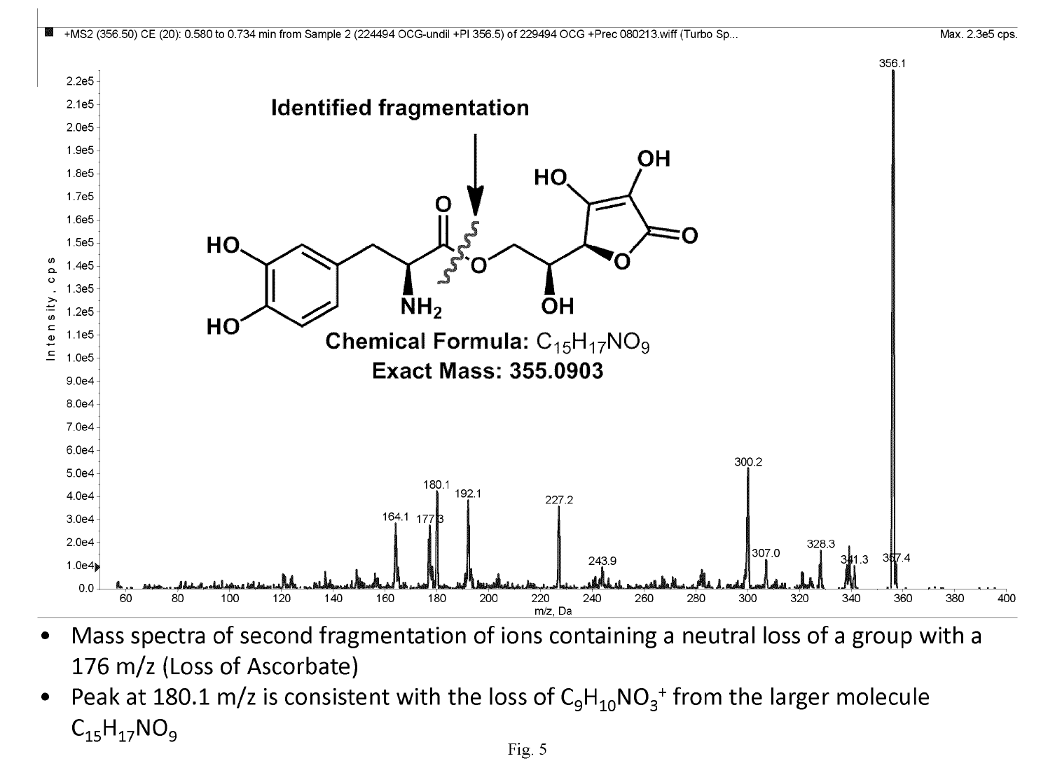

An experiment was designed to confirm the presence of ascorbate conjugates in isolated trabecular meshwork tissue samples from non-glaucomatous donors. Tandem mass spectrometry (MS/MS) and liquid chromatography-tandem mass spectrometry (LC/MS/IMS) techniques were developed using 6-O-Palmitoyl-L-Ascorbic acid as surrogate for the proposed detection of ascorbate conjugates. Provided eye tissues were then prepared for MS analysis. Samples were screened for mechanistic ester linked ascorbate structures through precursor ion scanning techniques. To provide screening procedures for the possible discovery of these molecules in eye tissue slices, standard molecules were studied to optimize their detection through neutral loss MS/MS detection from a reversed phase HPLC separation. These neutral loss detection experiments were centered around the loss of 176.1 u for ascorbate sugars. The figure of 176.1 u was determined by looking at proposed structures of ascorbate conjugates 204 for tyrosine 206 and L-DOPA 208 as shown in FIG. 3. Ascorbic acid 200 has the chemical formula C₆H₈O₆ with an exact mass of 176.03 and a molecular weight of 176.12. The ascorbate conjugate fragmentation (FIG. 4) of an ascorbate conjugate with formula C₁₅H₁₇NO₈ with an exact mass of 339.10 and a molecular weight of 339.30 was proposed to occur, leaving a tyrosine group with formula C₉H₁₀NO₂⁺ and an exact mass of 164.07 and the ascorbate molecule with formula C₆H₇O₆^{.} with an exact mass of 175.02. Alternatively, the ascorbate conjugate fragmentation (FIG. 5) of the ascorbate conjugate with formula C₁₅H₁₇NO₉ with an exact mass of 355.09 and a molecular weight of 355.30 was proposed to occur, leaving a L-DOPA group with formula C₉H₁₀NO₃⁺ and an exact mass of 180.07 and the ascorbate molecule with formula C₆H₇O₆^{.} with an exact mass 175.02.

Corneal tissue with scleral rims were stored in tissue culture media and refrigerated at 2-8° C. until initial extraction was performed. The samples were extracted as follows: the tissue was rinsed in balanced salt solution and the trabecular mesh was stripped off using microsurgical instruments and a dissecting microscope. The tissue was transferred to a 13×100 mm glass test tube. The eye tissue was ground with the end of a glass stirring rod. Tissue was noted to be very fibrous and resistant to disruption. 1.0 mL of HPLC grade methanol was added to the tissue and mixed. The mixture was then sonicated in 37° C. water bath for 1 hour. Next, 1.0 ml of HPLC grade chloroform was added and mixed for 2 minutes on high setting. The mixture was then centrifuged at 2500 rpm for 5 minutes. Next, the bottom layer was transferred to a clean 13×100 mm glass test tube. The top layer was re-extracted with an additional 1.0 mL of HPLC grade chloroform. The mixture was centrifuged as before and resultant lower layer combined with initial lower layer. The chloroform was evaporated to dryness and the resultant material reconstituted with 100 μL of HPLC mobile phase B.

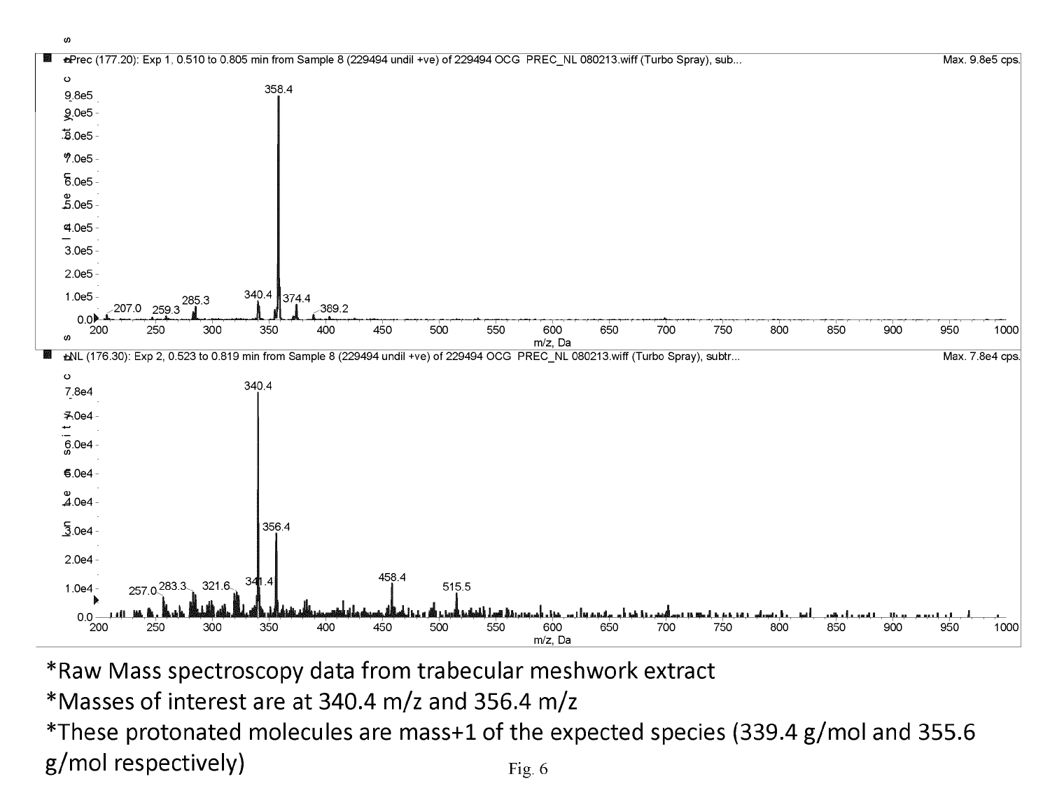

The samples were then analyzed via LC/+NL 176.1 u scan (FIG. 6), The positive ions did not fragment to the characteristic NL 158 u ion of ascorbic acid. The negative ions did not fragment to the characteristic NL 157 u ion of ascorbic acid.

The calculated ligand molecular weight for positive ions 358.4 u and 374.4 u were 199.1 u and 215.1 u, respectively. These molecular weight values were approximately 16 u apart, which suggests a difference in structure of an OH group.

Positive ions 358.4 u and 374.4 u were approximately 18 u apart from positive ions 340.4 u and 356.4 u. Positive ions 340.4 u and 356.4 u had molecular weights of 181.2 u and 196.3 u, respectively. This suggests a loss of an H₂O group or a loss of a (NH₄)⁺ group.

The calculated ligand molecular weight for negative ions 337.3 u and 369 u were 180.4 u and 212.4 u, respectively. These molecular weight values were 32 u apart, which suggests a difference in structure of 2(OH) groups.

From these data observations, it was determined that the molecular weight of the positive 340.4 u ion was 339.4 u. Accordingly, the ligand molecular weight was calculated by subtracting the NL 158 u ion of ascorbic acid from the 339.4 u molecular weight of the positive 340.4 u ion, which yielded a ligand molecular weight of 181.4 u.

The molecular weight of the positive 356.4 u ion was 355.4 u. Subtracting the NL 158 u ion of ascorbic acid from the 355.4 u molecular weight of the positive 356.4 u ion yielded a ligand molecular weight of 197.4 u.

The molecular weight of the negative 337.3 u ion was 338.3 u. Subtracting the NL 158 u ion of ascorbic acid from the 338.3 u molecular weight of the negative 337.3 u ion yielded a ligand molecular weight of 180.3 u.

The molecular weight of the negative 369.0 u ion was 370.0 u. Subtracting the NL 158 u ion of ascorbic acid from the 370.0 u molecular weight of the 369.0 u ion yielded a ligand molecular weight of 212.0 u.

Proposed ascorbate conjugates and fragments of tyrosine and L-DOPA were assayed under LC/MS/MS to corroborate the ligand identities by monitoring the fragmentation of 340.4 u and 356.4 u.

### Proposed Mechanism

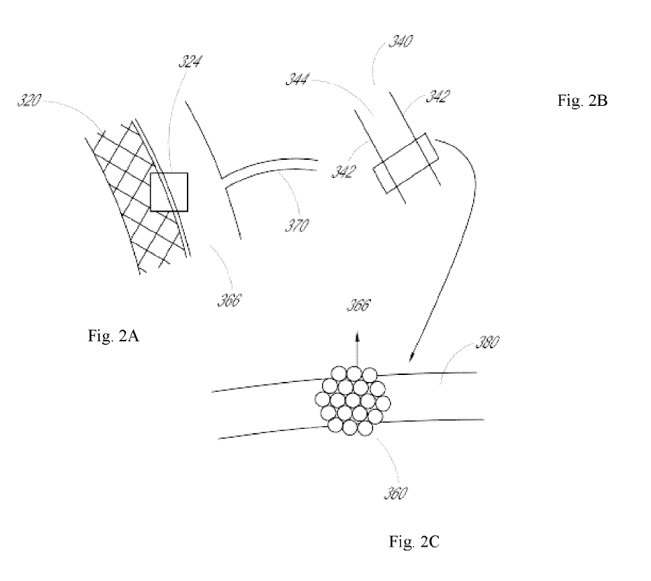

The experiment shows the presence of ascorbate as a component of tyrosine or L-DOPA in samples of eye tissue. Without wanting to be bound by any theory, it is believed that tyrosine or L-DOPA associated with an ascorbic acid or derivative or salt thereof are present in normal eye tissue, specifically in the endothelial layer 324 of Schlemm's canal 366 and/or the trabecular meshwork cells, and may play a role in the maintenance of normal intraocular pressure by regulating drainage of the aqueous humor through the membranes of the JCM cells as part of an osmotic drive. As seen in FIG. 2A, the trabecular meshwork 320 is separated from Schlemm's canal 366 by a single layer of endothelial cells 324. Once the aqueous humor passes through the endothelial layer, it drains into Schlemm's canal and then into the scleral venous system by way of bridging vessels 370. In FIG. 2B, the cell membrane 340 of an endothelial cell is seen with lipophilic regions 342 and hydrophilic regions 344. FIG. 2C shows the endothelial cell membrane 380 with direction of aqueous humor travel indicated by the arrow facing Schlemm's canal 366. A proposed structure in the cell membrane of the endothelial layer 360 is shown as an arrangement of micelles 366, which bridge the cell membrane 340, and serve to transport aqueous humor across the membrane. After traversing the opposing membrane, the aqueous humor is released into Schlemm's canal.

It is proposed that tyrosine molecules or L-DOPA molecules comprising an ascorbic acid or ascorbic acid derivative head are produced by the specialized cells of the JCM and transported to the cell membranes, where they bond to transmembrane proteins. Alternatively, the ascorbic acid or ascorbic acid derivative head may also be bound to an —OH group or other functional group of an amino acid such as tyrosine or L-DOPA already incorporated in a protein. A transmembrane cylinder may be formed with enough protein molecules such that a hydrophilic interior is formed for water and solute transport. When intraocular pressure is low, the ascorbic acid heads form bonds to each other, and as the intraocular pressure rises, the cellular osmolarity drops and cell volume increases, placing the cell membrane on stretch. This allows the water molecules to compete increasingly effectively at the binding sites, thereby allowing water to pass through the channel. The increasing pressure from surrounding aqueous fluid may also play a mechanical role in distorting the cell membrane, thereby contributing to the dissociation of bonds between polar moieties and the consequent permeability to water molecules. Mechanical forces may also initiate pinocytotic vesicle formation that has been observed in the JCM and the inner wall of Schlemm's Canal. As the intraocular pressure diminishes in response to increased flow, the bonds between polar moieties are increasingly favored over bonds with water molecules, and the flow diminishes, until an equilibrium is reached. The equilibrium may change based on various factors, such as the rate of production of aqueous humor, but will be self-regulating to maintain a desired pressure.

The above-described conjugates, spanning the cell membrane, may result in a self-regulating osmotic drive for water transport out of the anterior chamber of the eye into Schlemm's canal. When the pressure is balanced, the ascorbic acid moieties will generally bond with each other and water molecules from the aqueous humor will transport between the hydrophilic heads relatively slowly at a steady state rate. However, even small increases or decreases in pressure may cause the establishment of a new equilibrium flow rate.

Open angle glaucoma may result with a failure in the osmotic drive above. As relatively normal concentrations of ascorbic acid have been found to be present in eye tissue of glaucoma patients, absorption, transport, and ingestion of ascorbic acid are not likely causes of failure, and, furthermore would be expected to cause systemic problems related to vitamin C deficiency rather than isolated intraocular pressure elevations. In some patients, failure of normal conjugation of ascorbic acid with tyrosine or L-DOPA may result from enzyme deficiency, decreased enzyme activity, or other disturbance. These enzymes may be specific to the cells of the eye or JCM or may exist in other places, in which case the patient may have other manifestations in addition to glaucoma, and therapeutic molecules may treat those manifestations as well. In some patients, other pathologies may also result in the inability of the osmotic drive to assemble within the cell membrane.

### Therapeutics

The molecules disclosed herein may be employed as pharmaceutical agents, provided in therapeutically effective amounts, to effect the treatment of diseases and conditions, particularly open angle glaucoma. The term “treat,” “treating,” or “treatment” as used herein refers to administering a molecule or pharmaceutical composition to a subject for prophylactic and/or therapeutic purposes, and includes: (i) preventing a disease from occurring in a subject which may be predisposed to the disease but has not yet been diagnosed as having it; (ii) inhibiting the disease, i.e. arresting its development, or (iii) relieving the disease, i.e. causing regression of the disease.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

As used herein, the term “therapeutically effective amount,” “pharmaceutically effective amount” or “effective amount” refers to that amount (at dosages and for periods of time necessary) of a molecule which, when administered to a mammal in need thereof, is sufficient to effect treatment (as defined above). The amount that constitutes a “therapeutically effective amount” will vary depending on the molecule being administered, the condition or disease and its severity, and the mammal to be treated, its weight, age, etc., but may be determined routinely by one of ordinary skill in the art with regard to contemporary knowledge and to this disclosure.

### Molecules and Other Ascorbic Acid Deriatives

In some embodiments, an individual suffering from glaucoma is treated by administering a therapeutically effective amount of a therapeutic molecule which consists of an ascorbic acid conjugate, comprising ascorbic acid or a derivative thereof conjugated to a hydrophilic or amphipathic moiety. In some embodiments, the hydrophilic or amphipathic moiety comprises tyrosine or L-DOPA, or derivatives thereof. In another embodiment, an individual suffering from glaucoma may be treated by administering a therapeutically effective amount of a therapeutic molecule which can be configured to enhance the affinity of ascorbic acid or an ascorbic acid derivative to the hydrophilic or amphipathic moiety. In some embodiments, the hydrophilic or amphipathic moiety comprises tyrosine or an analog thereof. In some embodiments, the hydrophilic or amphipathic moiety comprises L-DOPA or an analog thereof. The ascorbate is not limited with respect to its form, and any known ascorbate or ascorbate derivative can be used. For example, ascorbate, ascorbic acid, or any pharmaceutically acceptable salt, hydrate, and solvate thereof, can be linked to the tyrosine or L-DOPA group and delivered to a patient in therapeutically effective amounts. Other polar molecules that have a single or multiple sites capable of hydrogen bonding may also be substituted for the ascorbate group. Disclosed below are possible ascorbic acid conjugates, comprising ascorbic acid conjugated to tyrosine or L-DOPA, wherein the R moiety represents tyrosine (R═—H) or L-DOPA (R═—OH). Also disclosed below are possible substitution patterns for ascorbic acid conjugated to tyrosine or L-DOPA. Examples of the R moiety include, but are not limited to, —H, —HCO, —H₃CCO, —(CH₃)₂HCCO and —(CH₃)₃CCO. Examples of the X moiety include, but are not limited to, —H, —F, —Cl, —Br, —I, —OH and —OR₁. Synthetic variations of the ascorbic acid conjugates disclosed herein may also be made.

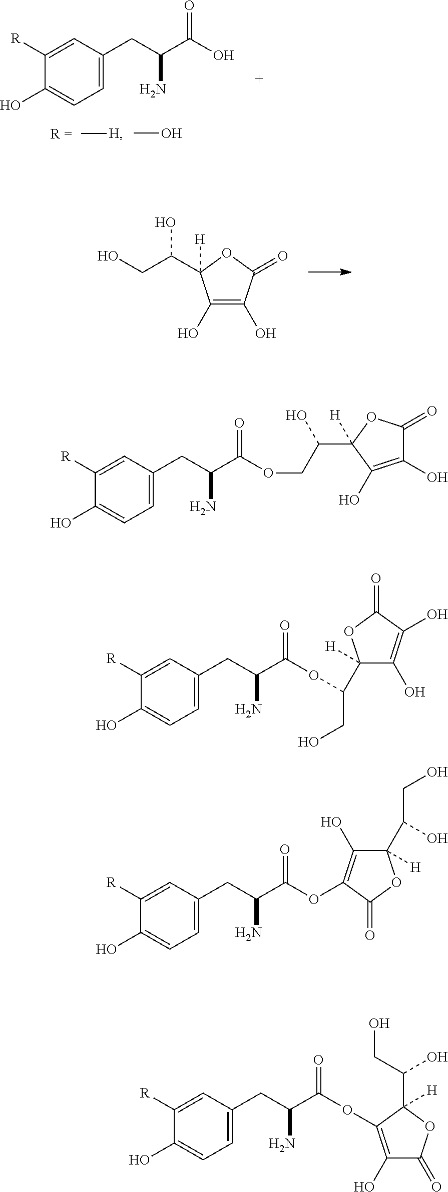

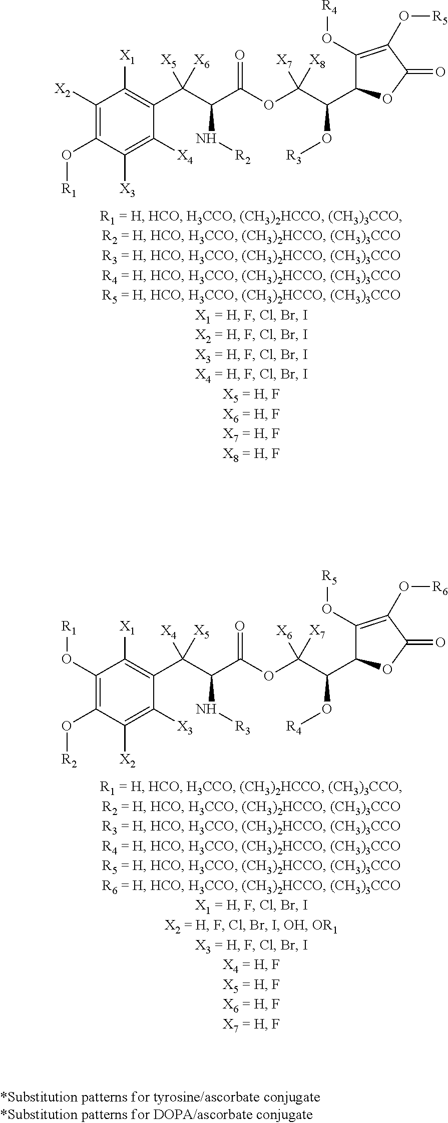

“Solvate” refers to the molecule formed by the interaction of a solvent and a molecule described herein or salt thereof; suitable solvates are pharmaceutically acceptable solvates including hydrates.

The term “pharmaceutically acceptable salt” refers to salts that retain the biological effectiveness and properties of a molecule and, which are not biologically or otherwise undesirable for use in a pharmaceutical. In many cases, the molecules disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

### Enzyme

In other embodiments, an enzyme having an enzymatic activity of forming a molecule comprising ascorbic acid or pharmaceutically acceptable derivative thereof conjugated to a ligand is administered in therapeutically effective amounts for uptake into the eye. In other embodiments, an enzyme having an enzymatic activity of conjugating ascorbic acid or pharmaceutically acceptable derivative thereof to a ligand is administered in therapeutically effective amounts for uptake into the eye. In some embodiments, an enzyme having an enzymatic activity of linking an ascorbic acid conjugate, comprising ascorbic acid or a derivative thereof conjugated to a ligand, to a transmembrane protein, as part of the construction of a transmembrane pore, is administered in therapeutically effective amounts for uptake into the eye. In some embodiments, the ligand may be tyrosine or an analog thereof. In some embodiments, the ligand may be L-DOPA or an analog thereof. In some embodiments, the enzyme may be a kinase that is capable of phosphorylating ascorbate. For example, phospholipase D can be used to synthesize 6-Phosphatidyl-L-ascorbic acid as described by Nagao et al. in Lipids 26:390-94 (1991), herein incorporated by reference in its entirety. Phospholipase D from Streptomyces lydicus may be obtained or the enzyme may be synthesized in a lab, both of which can be accomplished via methods known in the art. Other enzymes which are effective for phosphorylating ascorbate may be synthesized or isolated and administered to a subject in therapeutically effective amounts.

In some embodiments, the enzyme may be an esterase. In some embodiments, the esterase can be administered intracamerally by passing a blade, needle, applicator, or delivery system through the cornea. In other embodiments, the esterase can be applied directly onto the trabecular meshwork. As a non-limiting example, the trabecular meshwork can be contained in a paste-like erodible carrier under direct viewing by gonioscopy. The enzyme may be obtained from transgenetic subjects, such as chickens. Treatments directed at using transgenetic subjects have been proposed and approved by the United States Food and Drug Administration (e.g. Kanuma (sebelipase alfa), http://www.fda.gov/NewsEvents/Newsroom/PressAnnouncements/ucm476013.htm, herein incorporated by reference in its entirety).

Of course other enzymes besides esterases may be implicated. The present disclosure is not limited to the particular enzyme. As specific enzyme defects in the trabecular meshwork cells and Canal of Schlemm endothelial cells are identified, which enzyme defects are related to aqueous outflow pathways of the eye, those enzymes will also be potential therapeutic targets within the scope of the disclosure. For example, the enzyme classes may include phosphatases, phosphodiesterases, nucleases, proteases, transferases, ribosomal and non-ribosomal synthetases, and other enzymes that catalyze condensation reactions (e.g., amide- and ester-forming condensation enzymes), etc.

### Gene Therapy

In still other embodiments, treatment consists of gene therapy, in which one or more of the therapeutic agents is a nucleic acid that encodes a therapeutic agent. For example, the nucleic acid may encode for a protein or peptide. The protein or peptide may comprise an enzyme having an enzymatic activity of conjugating ascorbic acid or pharmaceutically acceptable derivative thereof to a ligand. In some embodiments, the protein or peptide may comprise an enzyme having an enzymatic activity of linking an ascorbic acid conjugate, comprising ascorbic acid or a derivative thereof conjugated to a ligand, to a transmembrane protein, as part of the construction of a transmembrane pore. In some embodiments, the ligand may be tyrosine or an analog thereof. In some embodiments, the ligand may be L-DOPA or an analog thereof. The protein or peptide may comprise a functional kinase enzyme to phosphorylate ascorbic acid, or to phosphorylate an ascorbate derivative or otherwise contribute to the production of ascorbic acid, ascorbate derivate or ascorbate equivalent conjugates in operable association with regulatory elements sufficient to direct expression of the nucleic acid administered to the eye. As another non-limiting example, the nucleic acid may encode a protein or peptide having esterase activity, wherein the functional esterase enzyme may cleave ester groups and release conjugate molecules. A composition comprising a nucleic acid therapeutic can consist essentially of the nucleic acid or a gene therapy vector in an acceptable diluent, or can comprise a drug release regulating component such as a polymer matrix with which the nucleic acid or gene therapy vector is physically associated; e.g., with which it is mixed or within which it is encapsulated or embedded. The gene therapy vector can be a plasmid, virus, or other vector. Alternatively, the pharmaceutical composition can comprise one or more cells which produce a therapeutic nucleic acid or polypeptide. Preferably such cells secrete the therapeutic agent into the extracellular space.

Alternatively, because the eye is a relatively immune privileged site, this allows for the successful transplantation of donor corneas without tissue typing or immunosuppression. Accordingly, cellular therapy can be effective. This immune privileged status may be used to administer donor trabecular meshwork cells to subjects to lower intraocular pressure. In some embodiments the trabecular meshwork cells may be grown in vitro, e.g., in tissue culture, suspension culture, organ culture, etc. In other embodiments, the trabecular meshwork cells may be harvested from eye bank tissue. In some embodiments, the trabecular meshwork cells may be xenogenic, allogenic or autogenic. In some embodiments, the trabecular meshwork cells can be administered in free solution. In some embodiments, the trabecular meshwork cells can be administered intracamerally by passing a blade, needle, applicator, or delivery system through the cornea. The trabecular meshwork cells can be released into the anterior chamber of the eye, where the aqueous drainage will carry them into the trabecular meshwork. See e.g., Yue et al. (1988) Monkey trabecular meshwork cells in culture: growth, morphologic, and biochemical characteristics. Graefes Arch Clin Exp Opthalmol., 226(3): 262-268; Russell et al. (2008) Response of human trabecular meshwork cells to topographic cues on the nanoscale level. Invest Opthalmol Vis Sci 49(2): 629-635; Gasiorowski and Russell (2009) Biological properties of trabecular meshwork cells. Exp Eye Res 88(4): 671-675; all of the above cited references are incorporated herein in their entireties by reference thereto.

Trabecular meshwork cells can be administered onto a trabecular meshwork of an eye to lower intraocular pressure. In some embodiments the trabecular meshwork cells may be grown in tissue cultures. In some embodiments, the trabecular meshwork cells can be applied directly onto the trabecular meshwork. In some embodiments, the trabecular meshwork cells can be administered under indirect visualization. In other embodiments, the trabecular meshwork cells can be administered under direct visualization. As a non-limiting example, the trabecular meshwork can be contained in a paste-like erodible carrier under direct viewing by gonioscopy.

In some embodiments, if it is necessary to repair a patient's own cells, genome engineering technologies, e.g., based on the CRISPR-associated RNA-guided endonuclease Cas9 may be used to repair a hereditary or acquired defect in trabecular meshwork cells grown in tissue culture media. These may be the patient's own trabecular meshwork cells or perhaps modified cells from the patient. The patient's trabecular meshwork cells can be removed as a strip for cell culture without harm to the patient. One glaucoma surgery, Trabectome, is based on this and morbidities are low. To determine the actual dysfunctional nucleic acid sequences, trabecular meshwork is easily stripped from corneal rims, cultured, and the genome cataloged. The same can be done for eyes from a patient with open angle glaucoma, with the defective nucleic acid sequence identified for targeting with genome engineering technologies e.g., based on the CRISPR-associated RNA-guided endonuclease Cas9. The repaired cell may then be reintroduced to the patient using the previously mentioned techniques. For detailed methods of gene editing using CRISPR technology, see e.g., Doudna and Charpenteir (2014) The new frontier of genome engineering with CRISPR-Cas9. Science Vol. 346 no. 6213; Long et al. (2014) Prevention of muscular dystrophy in mice by CRISPR/Cas9-mediated editing of germline DNA. Science Vol. 345 no. 6201 pp. 1184-1188; Slaymaker et al. (2015) Rationally engineered Cas9 nucleases with improved specificity. Science DOI: 10.1126/science.aad5227; all of the above cited references are incorporated herein in their entireties by reference thereto.

Viral vectors that have been used for gene therapy protocols include, but are not limited to, retroviruses, lentiviruses, other RNA viruses such as poliovirus or Sindbis virus, adenovirus, adeno-associated virus, herpes viruses, SV 40, vaccinia and other DNA viruses. Replication-defective murine retroviral or lentiviral vectors are widely utilized gene transfer vectors. Chemical methods of gene therapy involve carrier-mediated gene transfer through the use of fusogenic lipid vesicles such as liposomes or other vesicles for membrane fusion. A carrier harboring a nucleic acid of interest can be conveniently introduced into the eye or into body fluids or the bloodstream. The carrier can be site specifically directed to the target organ or tissue in the body. Cell or tissue specific DNA-carrying liposomes, for example, can be used and the foreign nucleic acid carried by the liposome absorbed by those specific cells. Gene transfer may also involve the use of lipid-based molecules which are not liposomes. For example, lipofectins and cytofectins are lipid-based molecules containing positive ions that bind to negatively charged nucleic acids and form a complex that can ferry the nucleic acid across a cell membrane.

Delivery of gene therapy may also be accomplished via cationic polymers. Certain cationic polymers spontaneously bind to and condense nucleic acids such as DNA into nanoparticles. For example, naturally occurring proteins, peptides, or derivatives thereof have been used. Synthetic cationic polymers such as polyethylenimine (PEI), polylysine (PLL) etc. condense DNA and are useful delivery vehicles. Dendrimers can also be used. Many useful polymers contain both chargeable amino groups, to allow for ionic interaction with the negatively charged DNA phosphate, and a degradable region, such as a hydrolyzable ester linkage. Examples include poly(alpha-(4-aminobutyl)-L-glycolic acid), network poly(amino ester), and poly (beta-amino esters). These complexation agents can protect nucleic acids against degradation, e.g., by nucleases, serum components, etc., and create a less negative surface charge, which may facilitate passage through hydrophobic membranes (e.g., cytoplasmic, lysosomal, endosomal, nuclear) of the cell. Certain complexation agents facilitate intracellular trafficking events such as endosomal escape, cytoplasmic transport, and nuclear entry, and can dissociate from the nucleic acid.

### Individual Treatment

In some embodiments, the treatment of glaucoma is tailored to the individual patient. Multiple variations of the molecule with different concentrations of tyrosine, L-DOPA, ascorbic acid or respective analogs and pharmaceutically acceptable derivatives thereof are provided, wherein administration of each variation results in characteristic reduction of intraocular pressure or a characteristic pressure at equilibrium. Methods of treatment may include the measurement of intraocular pressure prior to administration of the therapeutic agent, selection of molecule based on the desired reduction in intraocular pressure or target pressure, and administration of that molecule. The intraocular pressure may be monitored during therapy and different agents or a combination of different agents may be selected to maintain a desired pressure; for example, between 10 and 20 mm Hg, or sometimes between about 15 to about 18 mm Hg. After the selection of such different agents or combination of different agents, they may then be administered.

### Methods of Administration

Molecules or their precursors that increase transport of aqueous humor may be modified in an effort to increase the ability of the molecule to enter the eye. Examples may include, but are not limited to, the addition of cleavable ester groups or other easy leaving groups and molecules/compounds alterable by native enzymes or metabolic pathways into the intended molecules capable of increasing transport of aqueous humor.

Various methods of administering the therapeutic molecules systematically are contemplated. These include topical administration to the eye via drops, spray, gel, ointment, or other vehicle. The active molecules disclosed herein are administered to the eyes of a patient by any suitable means, but preferably administered by administering a liquid or gel suspension of the active molecule in the form of drops, spray or gel. Alternatively, the active molecules are applied to the eye via liposomes. Further, the active molecules can be infused into the tear film via a pump-catheter system. Another embodiment involves the therapeutic molecule contained within a continuous or selective-release device, for example, membranes such as, but not limited to, those employed in the Ocusert™ System (Alza Corp., Palo Alto, Calif.). As an additional embodiment, the active molecules can be contained within, carried by, or attached to contact lenses, which are placed on the eye. Another embodiment of the invention involves the therapeutic molecule contained within a swab or sponge, which is applied to the ocular surface. Another embodiment of the invention involves the therapeutic molecule contained within a liquid spray, which is applied to the ocular surface.

In other embodiments, the therapeutic molecule is delivered by intraocular injection performed periodically. In some embodiments, the therapeutic molecules may be administered via subconjunctival injection, in others through intracameral (anterior chamber), intravitreal or subscleral injection. The therapeutic molecule may be delivered directly to Schlemm's canal via catheter or implanted shunt. Further means of systemic administration of the active molecule would involve direct intra-operative instillation of a gel, cream, or liquid suspension form of a therapeutically effective amount of the therapeutic molecule. In some embodiments, the therapeutic molecules are administered in a suspension. In some embodiments, the therapeutic molecules may be administered, for example, by sustained release implants and microspheres for intracameral or anterior vitreal placement within a biodegradable polymer that releases a therapeutic amount of the molecule over a period of time ranging up to a year or more. Additionally, in some embodiments, the therapeutic molecules may be administered by an implanted drug delivery system which releases a therapeutically effective amount of the molecule over time. Implantation of the drug delivery system may be surgical or via injection. In some embodiments, the therapeutic molecule is delivered by iontophoresis. In some embodiments, the therapeutic molecule is delivered by ultrasound.

The topical solution containing the therapeutic molecule can also contain a physiologically compatible vehicle, as those skilled in the ophthalmic art can select using conventional criteria. The vehicles can be selected from the known ophthalmic vehicles which include, but are not limited to, saline solution, water polyethers (such as polyethylene glycol), polyvinyls (such as polyvinyl alcohol and povidone), cellulose derivatives (such as methylcellulose and hydroxypropyl methylcellulose), petroleum derivatives (such as mineral oil and white petrolatum), animal fats (such as lanolin), polymers of acrylic acid (such as carboxypolymethylene gel), vegetable fats (such as peanut oil) and polysaccharides (such as dextrans), and glycosaminoglycans (such as sodium hyaluronate), and salts (such as sodium chloride and potassium chloride). In some embodiments, the pH of the topical solution containing the therapeutic molecule can be adjusted to a pH of about 7 to about 11. In some embodiments, the pH can be about 7, about 8, about 9, about 10, about 11, or a range between any two of these values. In some embodiments, the therapeutic molecule for topical administration can be less than or equal to about 500 Daltons, as described or modified from Jan D. Bos, et al., “The 500 Dalton rule for the skin penetration of chemical compounds and drugs,” Exp Dermatol. 2000; 9(3):165-9, which is herein incorporated by reference in its entirety.

In addition to the topical method of administration described above, there are various methods of administering the therapeutic molecules of the invention systemically. One systemic method of administration may involve an aerosol suspension of respirable particles comprised of the active molecule, which the subject inhales. The therapeutic molecule is absorbed into the bloodstream via the lungs and subsequently contact the ocular tissues in a pharmaceutically effective amount. The respirable particles are a liquid or solid, with a particle size sufficiently small to pass through the mouth and larynx upon inhalation; in general, particles ranging from about 1 to 10 microns, but more preferably 1-5 microns, in size are considered respirable.

Another means of systemically administering the active molecules to the eyes of the subject would involve administering a liquid/liquid suspension in the form of eye drops or eye wash or nasal drops of a liquid formulation, or a nasal spray of respirable particles which the subject inhales. Liquid pharmaceutical compositions of the active molecule for producing a nasal spray or nasal or eye drops can be prepared by combining the active molecule with a suitable vehicle, such as sterile pyrogen free water or sterile saline by techniques known to those skilled in the art.

Other means of systemic administration of the therapeutic molecule may involve oral administration, in which pharmaceutical compositions containing active molecules are in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate: granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glvceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Additional means of systemic administration of the therapeutic molecule to the eyes of the subject would involve a suppository form of the active molecule, such that a therapeutically effective amount of the molecule reaches the eyes via systemic absorption and circulation.

### EXAMPLES

### Example 1— Treatment of Glaucoma Using Ascorbic Acid Linked to Tyrosine [Prophetic]

A 75 year old patient presents with intraocular pressure (IOP) of 26 mm Hg in both eyes. This represents 4 standard deviations (2.5 mmHg) above average IOP (16 mm Hg). The filtering angles are inspected by gonioscopy and found to be open. Inspection of the nasal and temporal portions of the eye where aqueous veins are most prominent show the structures to be intact. The optic nerves show only moderate damage. This patient would not have very low target IOP's and normalization of IOP would represent adequate response to therapy. The expected therapy regimen would be a loading dose of eye drops 2-3 times per day comprising up to 10 mg ascorbic acid-tyrosine conjugate in a vehicle or delivery system. After 2 weeks on the treatment regimen, the intraocular pressure would be reduced and the medication cut to twice daily. At 1 month, once the intraocular pressure is normalized, the drops may be tapered to the minimum required to maintain a therapeutic effect.

Although embodiments and methods have been disclosed in the context of glaucoma treatment, it will be understood by those skilled in the art that embodiments and methods disclosed herein may also be used in other contexts. For example, administration of ascorbic acid linked to tyrosine or L-DOPA may be utilized in therapeutic amounts to treat other disorders in which fluid outflow regulation is dysfunctional. Examples in which fluid outflow regulation utilize disclosed therapeutic methods include, but are not limited to, the treatment of hydrocephalus and in an artificial kidney.

Although this has been disclosed in the context of certain preferred embodiments and examples, it will be understood by those skilled in the art that the invention extends beyond the specifically disclosed embodiments to other alternative embodiments and/or uses of the invention and obvious modifications and equivalents thereof. In addition, while the number of variations of the invention have been shown and described in detail, other modifications, which are within the scope of this invention, will be readily apparent to those of skill in the art based upon this disclosure. It is also contemplated that various combinations or subcombinations of the specific features and aspects of the embodiments can be made and still fall within the scope of the invention. Accordingly, it should be understood that various features and aspects of the disclosed embodiments can be combined with, or substituted for, one another in order to perform varying modes of the disclosed invention. Thus, it is intended that the scope of the invention herein disclosed should not be limited by the particular disclosed embodiments described above, but should be determined only by a fair reading of the claims.

In this application, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments. Additionally, in this application, “and/or” denotes that both the inclusive meaning of “and” and, alternatively, the exclusive meaning of “or” applies to the list. Thus, the listing should be read to include all possible combinations of the items of the list and to also include each item, exclusively, from the other items. The addition of this term is not meant to denote any particular meaning to the use of the terms “and” or “or” alone. The meaning of such terms will be evident to one of skill in the art upon reading the particular disclosure.

All references cited herein including, but not limited to, published and unpublished patent applications, patents, text books, literature references, and the like, to the extent that they are not already, are hereby incorporated by reference in their entirety. To the extent that one or more of the incorporated literature and similar materials differ from or contradict the disclosure contained in the specification, including but not limited to defined terms, term usage, described techniques, or the like, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

What is claimed is:

1. A method of lowering intraocular pressure, the method comprising administering to a subject in need of such treatment, a therapeutically effective amount of an ascorbic acid conjugate, wherein the ascorbic acid conjugate comprises ascorbic acid or a derivative thereof coupled to tyrosine or L-DOPA or derivatives thereof.

2. A method of lowering intraocular pressure, the method comprising administering to a subject in need of such treatment, a therapeutically effective amount of a composition that increases an enzymatic activity of conjugating ascorbic acid to tyrosine or a derivative thereof, or to L-DOPA or a derivative thereof.

3. The method of claim 2, wherein the composition is an enzyme or enzymatically active fragment thereof comprising kinase activity.

4. The method of claim 2, wherein the composition is an enzyme or enzymatically active fragment thereof comprising esterase activity.

5. The method of claim 2, wherein the composition is a nucleic acid that encodes for an enzyme or enzymatically active fragment thereof.

6. The method of claim 1, wherein administering further comprises implanting via surgery or injection a sustained release implant for intracameral or anterior vitreal placement.

7. A pharmaceutical composition for use in lowering intraocular pressure, the composition comprising ascorbic acid conjugated to tyrosine or L-DOPA or derivatives thereof, and a pharmaceutically acceptable carrier, wherein the pharmaceutical composition is configured for intraocular delivery.

8. A pharmaceutical composition for use in lowering intraocular pressure, the composition comprising an enzyme or enzymatically active fragment capable of catalyzing the conjugation of ascorbic acid to tyrosine or L-DOPA or derivatives thereof, and a pharmaceutically acceptable carrier, wherein the pharmaceutical composition is configured for intraocular delivery.

9. A pharmaceutical composition for use in lowering intraocular pressure, the composition comprising an enzyme or enzymatically active fragment capable of catalyzing the linking of an ascorbic acid conjugate to a transmembrane protein, and a pharmaceutically acceptable carrier, wherein the pharmaceutical composition is configured for intraocular delivery, and wherein the conjugate comprises ascorbic acid or a derivative thereof coupled to tyrosine or L-DOPA or derivatives thereof.

10. A pharmaceutical composition for use in lowering intraocular pressure, the composition comprising a nucleic acid which encodes an enzyme or enzymatically active fragment capable of catalyzing the conjugation of ascorbic acid to tyrosine or L-DOPA or derivatives thereof, and a pharmaceutically acceptable carrier, wherein the pharmaceutical composition is configured for gene therapy.

11. A pharmaceutical composition for use in lowering intraocular pressure, the composition comprising a nucleic acid which encodes an enzyme or enzymatically active fragment capable of catalyzing the linking of an ascorbic acid conjugate to a transmembrane protein, and a pharmaceutically acceptable carrier, wherein the pharmaceutical composition is configured for intraocular delivery, and wherein the conjugate comprises ascorbic acid or a derivative thereof coupled to tyrosine or L-DOPA or derivatives thereof.

12. A method of lowering intraocular pressure, the method comprising administering to a subject in need of such treatment, trabecular meshwork cells to an anterior chamber of an eye.

13. The method of claim 12, wherein the trabecular meshwork cells are administered in free solution.

14. A method of lowering intraocular pressure, the method comprising administering to a subject in need of such treatment, trabecular meshwork cells onto a trabecular meshwork of an eye.

15. The method of claim 14, wherein the trabecular meshwork cells are administered under direct visualization.

16. The method of claim 14, wherein the trabecular meshwork cells are administered under indirect visualization.

17. The method of any one of claims 14-16, wherein the trabecular meshwork cells are cultured cells from suspension, cell or organ cultures.

18. The method of any one of claims 14-17, wherein prior to administration the trabecular meshwork cells are genetically modified in vitro to correct a hereditary or acquired defect.

19. A composition for use in lowering intraocular pressure, the composition comprising trabecular meshwork cells in a pharmaceutically acceptable carrier, wherein the composition is configured for intraocular delivery or delivery to a region of the trabecular meshwork.
